(19) United States
(12) Patent Application Publication
(10) Pub. No.: US 2025/0319147 A1
(43) Pub. Date: Oct. 16, 2025

(54) COMPOSITIONS COMPRISING ECHINACEA EXTRACT

(71) Applicant: Shaman Naturals, LLC, Casper, WY (US)

(72) Inventor: Maria Crisler, Casper, WY (US)

(21) Appl. No.: 19/175,159

(22) Filed: Apr. 10, 2025

Related U.S. Application Data
(60) Provisional application No. 63/632,450, filed on Apr. 10, 2024.
(60) Provisional application No. 63/632,831, filed on Apr. 11, 2024.

(51) Int. Cl. A61K 36/28; A61K 9/00; A61K 31/198; A61K 31/352; A61K 31/375; A61K 33/04; A61K 33/30; A61K 38/06; A61K 47/10; A61K 47/24; A61P 3/10

(52) U.S. Cl. CPC A61K 36/28; A61K 9/006; A61K 31/198; A61K 31/352; A61K 31/375; A61K 33/04; A61K 33/30; A61K 38/063; A61K 47/10; A61K 47/24; A61P 3/10

(57) ABSTRACT

Disclosed herein are compositions comprising an Echinacea extract, a long chain alcohol, and a carrier. Also disclosed are methods of treating a disorder comprising administering to a subject in need thereof a therapeutically effective amount of any of the compositions disclosed herein. Also disclosed herein are kits for the treatment of a disease, comprising any of the compositions disclosed herein and instructions for administering the composition to a subject.

### CROSS-REFERENCE TO RELATED APPLICATIONS

This application claims the benefit of U.S. Provisional Application Ser. No. 63/632,450, filed on Apr. 10, 2024, and U.S. Provisional Application Ser. No. 63/632,831, filed on Apr. 11, 2024, each entitled “Compositions Comprising Echinacea Extract,” and each incorporated herein by reference in its entirety.

### TECHNICAL FIELD

The present disclosure relates to compositions comprising Echinacea extract for use in the treatment of diseases and disorders.

### BACKGROUND

Natural remedies for various diseases and disorders have been developed.

### SUMMARY

The present disclosure is directed to a composition comprising an Echinacea extract, a long chain alcohol, and a carrier.

The disclosure is further directed to a method of treating a disorder comprising administering to a subject in need thereof a therapeutically effective amount of any of the compositions disclosed herein.

The disclosure is further directed to a kit for the treatment of a disease, comprising any of the compositions disclosed herein and instructions for administering the composition to a subject.

### BRIEF DESCRIPTION OF THE DRAWINGS

FIG. 1 shows a graph of Patient 1's blood glucose levels as a function of time as measured in Example 1.

FIG. 2 shows a second graph of Patient 1's blood glucose levels as a function of time as measured in Example 1.

FIG. 3 shows a graph of Patient 1's blood glucose levels as a function of time over a 24-hour period, as measured in Example 2.

FIG. 4 shows a graph of Patient 2's blood glucose levels as a function of time as measured in Example 3.

FIG. 5 shows a graph of Patient 2's blood glucose levels as a function of time as measured in Example 4.

FIG. 6 shows a graph of Patient 2's blood glucose levels as a function of time as measured in Example 5.

FIG. 7 shows a second graph of Patient 2's blood glucose levels as a function of time as measured in Example 5.

FIG. 8 shows a graph of Patient 2's blood glucose levels as a function of time as measured in Example 6.

FIG. 9 shows a second graph of Patient 2's blood glucose levels as a function of time as measured in Example 6.

FIG. 10 shows a graph of Patient 3's blood glucose levels as a function of time as measured in Example 7.

### DETAILED DESCRIPTION

### Definitions

For purposes of the following detailed description, it is to be understood that the disclosure may assume various alternative variations and step sequences, except where expressly specified to the contrary.

The numerical values set forth in the specific examples are reported as precisely as possible. Any numerical value, however, inherently contains certain errors necessarily resulting from the standard variation found in their respective testing measurements.

Also, any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In addition, in this application, the use of “or” means “and/or” unless specifically stated otherwise, even though “and/or” may be explicitly used in certain instances.

As used herein, “including,” “containing,” and like terms are understood in the context of this application to be synonymous with “comprising” and are therefore open-ended and do not exclude the presence of additional undescribed and/or unrecited elements, materials, ingredients, and/or method steps.

As used herein, “consisting of” is understood in the context of this application to exclude the presence of any unspecified element, ingredient, and/or method step.

As used herein, “consisting essentially of” is understood in the context of this application to include the specified elements, materials, ingredients, and/or method steps “and those that do not materially affect the basic and novel characteristic(s)” of what is being described.

As used herein, the terms “patient,” “subject,” “individual,” and the like are used interchangeably herein and mean animals amenable to the methods of treatment described herein, including mammals, including a human, a canine, a feline, a bovine, an equine, a porcine, a primate, and/or a rodent.

As used herein, “administer,” “administering,” “administration” and like terms refer to dispensing or applying an amount (e.g., a dose) of a composition by the subject himself/herself or by another subject (e.g., a medical professional, a caretaker, or a family member).

As used herein, “disorder,” “disease,” and “illness” are used interchangeably and refer to a condition in a subject that negatively impacts the health of the subject.

As used herein, “treat,” “treatment,” “treating” and like terms mean a therapeutic, prophylactic, palliative, or preventative measure provided to a patient or subject with the intention of preventing the development or altering the pathology or symptoms experienced by the patient or subject, such as, e.g., those resulting from a disease, disorder, or illness. A “treatment” administered to a patient or subject may achieve any clinical or quantitatively measurable reduction in the condition for which the patient or subject is being treated up to and including complete elimination.

As used herein, a “therapeutically effective amount” is defined as an amount required to treat or prevent the symptoms of a disease, disorder, or illness in a treated patient relative to an untreated patient.

As used herein, “measuring” or “measurement” or alternatively “detecting” or “detection” means assessing the presence, absence, quantity, or amount (which can be any effective amount) of either a given substance within a clinical or a subject-driven sample, including the derivation of qualitative or quantitative levels of such substance, or otherwise evaluating the values or categorization of a subject's clinical parameters.

As used herein, “composition” means a solution, a dispersion, or a solid, such as a powder.

### Compositions

Disclosed herein is a composition comprising, consisting essentially of, or consisting of an Echinacea extract; a long chain alcohol; and a carrier.

The compositions of the present disclosure may comprise an Echinacea extract. Any suitable Echinacea extract known in the art may be used in the present disclosure, including but not limited to extracts from Echinacea purpurea, Echinacea angustifolia, and/or Echinacea pallida. The Echinacea extract may be derived from the roots, leaves, or flowers of an Echinacea plant.

The Echinacea extract may comprise bioactive compounds, including but not limited to alkylamides, glycoproteins, polysaccharides, flavonoids, and/or phenolic compounds. The phenolic compounds may comprise caffeic acid, echinacoside, chicoric acid, chlorogenic acid, and/or caftaric acid. The concentrations of the bioactive compounds may vary based upon the plant species and plant part from which the Echinacea extract is derived.

The composition of the present disclosure may comprise the Echinacea extract in an amount of at least 5 mg/mL, such as at least 10 mg/mL, such as at least 15 mg/mL. The composition of the present disclosure may comprise the Echinacea extract in an amount of no more than 35 mg/mL, such as no more than 30 mg/mL, such as no more than 25 mg/mL. The composition of the present disclosure may comprise the Echinacea extract in an amount of 5 mg/mL to 35 mg/mL, such as 10 mg/mL to 30 mg/mL, such as 15 mg/mL to 25 mg/mL.

The composition may comprise a long chain alcohol. The long chain alcohol may comprise a hydroxy functional group on a saturated, unsaturated, or polyunsaturated carbon chain. The carbon chain may comprise at least 20 carbons, such as at least 22 carbons, such as at least 24 carbons. The carbon chain may comprise no more than 40 carbons, such as no more than 36 carbons, such as no more than 34 carbons. The carbon chain may comprise 20 to 40 carbons, such as 20 to 36 carbons, such as 22 to 36 carbons, such as 22 to 34 carbons, such as 24 to 34 carbons. The hydroxy functional group may be a terminal group and/or a pendant group.

The long chain alcohol may comprise, consist essentially of, or consist of octacosanol, hexacosanol, triacontanol, docosanol, dotriacontanol, and/or tetracosanol.

The long chain alcohol may comprise octacosanol, hexacosanol, and/or triacontanol in a total amount of at least 40% by weight based on total weight of the long chain alcohol, such as at least 50% by weight, such as at least 60% by weight, such as at least 70% by weight, such as at least 80% by weight. The long chain alcohol may comprise octacosanol, hexacosanol, and/or triacontanol in a total amount of 100% by weight based on total weight of the long chain alcohol, such as no more than 95% by weight, such as no more than 90% by weight. The long chain alcohol may comprise octacosanol, hexacosanol, and/or triacontanol in a total amount of 40% by weight to 100% by weight based on total weight of the long chain alcohol, such as 40% by weight to 95% by weight, such as 40% by weight to 90% by weight, such as 50% by weight to 100% by weight, such as 50% by weight to 95% by weight, such as 50% by weight to 90% by weight, such as 60% by weight to 100% by weight, such as 60% by weight to 95% by weight, such as 60% by weight to 90% by weight, such as 70% by weight to 100% by weight, such as 70% by weight to 95% by weight, such as 70% by weight to 90% by weight, such as 80% by weight to 100% by weight, such as 80% by weight to 95% by weight, such as 80% by weight to 90% by weight.

The long chain alcohol may comprise octacosanol in an amount of at least 40% by weight based on total weight of the long chain alcohol, such as at least 45% by weight, such as at least 50% by weight, such as at least 55% by weight, such as at least 60% by weight. The long chain alcohol may comprise octacosanol in an amount of no more than 100% by weight based on total weight of the long chain alcohol, such as no more than 90% by weight, such as no more than 85% by weight, such as no more than 80% by weight, such as no more than 75% by weight, such as no more than 70% by weight. The long chain alcohol may comprise octacosanol in an amount of 40% by weight to 100% by weight based on total weight of the long chain alcohol, such as 40% by weight to 90% by weight, such as 45% percent by weight to 85% by weight, such as 50% by weight to 80% by weight, such as 55% by weight to 75% by weight, such as 60% by weight to 70% by weight.

The long chain alcohol may comprise hexacosanol in an amount of at least 5% by weight based on total weight of the long chain alcohol, such as at least 7% by weight, such as at least 10% by weight. The long chain alcohol may comprise hexacosanol in an amount of no more than 50% by weight based on total weight of the long chain alcohol, such as no more than 40% by weight, such as no more than 30% by weight, such as no more than 20% by weight, such as no more than 15% by weight. The long chain alcohol may comprise hexacosanol in an amount of 5% percent by weight to 50% by weight based on total weight of the long chain alcohol, such as 5% percent by weight to 40% by weight, such as 5% percent by weight to 30% by weight, such as 5% percent by weight to 20% by weight, such as 5% percent by weight to 15% by weight, such as 7% percent by weight to 50% by weight, such as 7% percent by weight to 40% by weight, such as 7% percent by weight to 30% by weight, such as 7% percent by weight to 20% by weight, such as 7% percent by weight to 15% by weight, such as 10% percent by weight to 50% by weight, such as 10% percent by weight to 40% by weight, such as 10% percent by weight to 30% by weight, such as 10% percent by weight to 20% by weight, such as 10% percent by weight to 15% by weight.

The long chain alcohol may comprise triacontanol in an amount of at least 5% by weight based on total weight of the long chain alcohol, such as at least 7% by weight, such as at least 10% by weight. The long chain alcohol may comprise triacontanol in an amount of no more than 50% by weight based on total weight of the long chain alcohol, such as no more than 40% by weight, such as no more than 30% by weight, such as no more than 20% by weight. The long chain alcohol may comprise triacontanol in an amount of 5% by weight to 50% by weight based on total weight of the long chain alcohol, such as 5% by weight to 40% by weight, such as 5% by weight to 30% by weight, such as 5% by weight to 20% by weight, such as 7% by weight to 50% by weight, such as 7% by weight to 40% by weight, such as 7% by weight to 30% by weight, such as 7% by weight to 20% by weight, such as 10% by weight to 50% by weight, such as 10% by weight to 40% by weight, such as 10% by weight to 30% by weight, such as 10% by weight to 20% by weight.

The long chain alcohol may comprise a weight ratio of hexacosanol to octacosanol of at least 1:19, such as at least 1:9, such as at least 1:5.5. The long chain alcohol may comprise a weight ratio of hexacosanol to octacosanol of no more than 1:1.5, such as no more than 1:2.5, such as no more than 1:4. The long chain alcohol may comprise a weight ratio of hexacosanol to octacosanol of 1:19 to 1:1.5, such as 1:9 to 1:2.5, such as 1:5.5 to 1:4.

The long chain alcohol may comprise a weight ratio of hexacosanol to triacontanol of at least 1:4, such as at least 1:2.5, such as at least 1:1.5. The long chain alcohol may comprise a weight ratio of hexacosanol to triacontanol of no more than 4:1, such as no more than 2.5:1, such as no more than 1.5:1, such as no more than 1:1. The long chain alcohol may comprise a weight ratio of hexacosanol to triacontanol of 1:4 to 4:1, such as 1:4 to 1:1, such as 1:2.5 to 2.5:1, such as 1:2.5 to 1:1, such as 1:1.5 to 1.5:1, such as 1:1.5 to 1:1.

The long chain alcohol may be in a powder form at ambient conditions. As used herein, “ambient conditions” refers to room temperature (e.g., 23° C.) and humidity conditions, e.g., at 10° C. to 40° C. and 5% to 80% relative humidity.

The long chain alcohol may have a bulk density of at least 0.3 g/mL as measured by USP 616, such as at least 0.35 g/mL, such as at least 0.4 g/mL. The long chain alcohol may have a bulk density of no more than 0.6 g/mL as measured by USP 616, such as no more than 0.55 g/mL, such as no more than 0.5 g/mL. The long chain alcohol may have a bulk density of 0.3 g/mL to 0.6 g/mL as measured by USP 616, such as 0.35 g/mL to 0.55 g/mL, such as 0.4 g/mL to 0.5 g/mL. As used herein, “bulk density” means the ratio of the mass of a material to its total volume, which includes the volume of the solid particles and the voids (spaces) between them.

The long chain alcohol may have a tapped density of at least 0.2 g/mL as measured by USP 616, such as at least 0.25 g/mL, such as at least 0.3 g/mL. The long chain alcohol may have a tapped density of no more than 0.8 g/mL as measured by USP 616, such as no more than 0.75 g/mL, such as no more than 0.7 g/mL. The long chain alcohol may have a tapped density of 0.2 g/mL to 0.8 g/mL as measured by USP 616, such as 0.25 g/mL to 0.75 g/mL, such as 0.3 g/mL to 0.7 g/mL. As used herein, “tapped density” means the mass of a powder per unit volume after the powder has been mechanically tapped to remove voids and air gaps between particles.

The long chain alcohol may be derived from any source, including but not limited to sugar, beeswax, cereal grains, grasses, leaves, fruits, buds, seeds, or combinations thereof.

The long chain alcohol may comprise nanoparticles. The long chain alcohol may be in the form of a nanoemulsion comprising nanoparticles. The nanoparticles may comprise a particle size of no more than 100 nm, such as a positive particle size up to 20 nm, such as up to 30 nm, such as up to 40 nm, such as up to 50 nm, such as up to 60 nm, such as up to 70 nm, such as up to 80 nm, such as up to 90 nm, such as up to 100 nm. Particle size may be measured by any method known to those skilled in the art, such as for example, using a scanning electron microscope (SEM). For example, powders may be dispersed on segments of carbon tape attached to aluminum stubs and coated with Au/Pd for 20 seconds. Samples then may be analyzed in an SEM under high vacuum (accelerating voltage 10 kV and spot size 3.0), measuring 30 particles from three different areas to provide an average particle size for each sample. One skilled in the art will recognize that there can be variations in this procedure that retain the essential elements of microscopic imaging and averaging of representative size. Alternatively, particle sizes may be reported by the manufacturer.

The nanoparticles may comprise a long chain alcohol and a stabilizer. The stabilizer may comprise, consist essentially of, or consist of polyethylene glycol ester, tocopheryl ester, tocopheryl polyethylene glycol ester, tocopheryl polyethylene glycol (1000) succinate (“TPGS”), or combinations thereof.

The concentration of the long chain alcohol may vary based upon, for example, the target organ or tissue, the disease indication being treated, and/or the projected length of time of treatment.

The composition may comprise the long chain alcohol in an amount of at least 5 mg/mL, such as at least 10 mg/mL, such as at least 15 mg/mL. The composition may comprise the long chain alcohol in an amount of no more than 35 mg/mL, such as no more than 30 mg/mL, such as no more than 25 mg/mL. The composition may comprise the long chain alcohol in an amount of 5 mg/mL to 35 mg/mL, such as 10 mg/mL to 30 mg/mL, such as 15 mg/mL to 25 mg/mL.

The composition may further comprise a carrier. As used herein, “carrier” refers to a solvent, diluent, or the like that is used to deliver the active agents in a composition. As used herein, an “active agent” refers to a component of a composition used to treat a patient in need thereof. The carrier may comprise, consist essentially of, or consist of a phospholipid and/or water. Suitable phospholipids include but are not limited to polyoxyethylene, sorbitan monolaurate, sorbitan monooleate, polyethylene glycol-hydrogenated castor oil (e.g., Cremophor and Cremophor RH), egg phospholipid, soy phospholipid, lecithin, such as sunflower lecithin, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, and/or coconut oil.

The composition may comprise the carrier in an amount of at least 600 mg/mL, such as at least 700 mg/mL, such as at least 800 mg/mL. The composition may comprise the carrier in an amount of no more than 1,500 mg/mL, such as no more than 1,400 mg/mL, such as no more than 1,300 mg/mL. The composition may comprise the carrier in an amount of 600 mg/mL to 1,500 mg/mL, such as 700 mg/mL to 1,400 mg/mL, such as 800 mg/mL to 1,300 mg/mL.

The carrier may comprise water. When water is present, the composition may comprise water in an amount of at least 200 mg/mL, such as at least 250 mg/mL, such as at least 300 mg/mL. The composition may comprise water in an amount of no more than 600 mg/mL, such as no more than 550 mg/mL, such as no more than 500 mg/mL. The composition may comprise water in an amount of 200 mg/mL to 600 mg/mL, such as 250 mg/mL to 550 mg/mL, such as 300 mg/mL to 500 mg/mL.

The phospholipid may comprise a first phospholipid, such as olive oil. The composition may comprise the first phospholipid in an amount of at least 400 mg/mL, such as at least 450 mg/mL, such as at least 500 mg/mL. The composition may comprise the first phospholipid in an amount of no more than 850 mg/mL, such as no more than 800 mg/mL, such as no more than 750 mg/mL. The composition may comprise the first phospholipid in an amount of 400 mg/mL to 850 mg/mL, such as 450 mg/mL to 800 mg/mL, such as 500 mg/mL to 750 mg/mL.

The phospholipid may comprise a second phospholipid, such as avocado oil. The composition may comprise the second phospholipid in an amount of at least 3 mg/mL, such as at least 4 mg/mL, such as at least 5 mg/mL. The composition may comprise the second phospholipid in an amount of no more than 15 mg/mL, such as no more than 12 mg/mL, such as no more than 10 mg/mL. The composition may comprise the second phospholipid in an amount of 3 mg/mL to 15 mg/mL, such as 4 mg/mL to 12 mg/mL, such as 5 mg/mL to 10 mg/mL.

The composition may further comprise an antioxidant in addition to the Echinacea extract. The antioxidant may comprise glutathione. The composition may comprise the antioxidant in an amount of at least 20 mg/mL, such as at least 25 mg/mL, such as at least 30 mg/mL. The composition may comprise the antioxidant in an amount of no more than 60 mg/mL, such as no more than 55 mg/mL, such as no more than 50 mg/mL. The composition may comprise the antioxidant in an amount of 20 mg/mL to 60 mg/mL, such as 25 mg/mL to 55 mg/mL, such as 30 mg/mL to 50 mg/mL.

The composition may further comprise an ionophore in addition to the Echinacea extract. As used herein, “ionophore” refers to a compound capable of transporting ions across a lipid membrane. To the extent the ionophore may act as an antioxidant, for purposes of this disclosure, the ionophore is treated as an additional component to the antioxidant. Suitable ionophores include but are not limited to a flavonoid, monensin, lasalocid, salinomycin, narasin, maduramicin, laidlomycin, semduramicin, pyrithione, and/or zincophorin. Suitable flavonoids include but are not limited to apigenin (4′,5,7-trihydroxyflavone), luteolin (3′,4′,5,7-tetrahydroxyflavone), tangeritin (4′,5,6,7,8-pentamethoxyflavone), chrysin (5,7-dihydroxyflavone), 6-hydroxyflavone, and/or quercetin (3,3′,4,4′,5,7-pentahydroxyflavone).

The ionophore may be present in the composition in an amount of at least 0.8 mg/mL, such as at least 1.0 mg/mL, such as at least 1.2 mg/mL. The ionophore may be present in the composition in an amount of no more than 2.4 mg/mL, such as no more than 2.2 mg/mL, such as no more than 2.0 mg/mL. The ionophore may be present in the composition in an amount of 0.8 mg/mL to 2.4 mg/mL, such as 1.0 mg/mL to 2.2 mg/mL, such as 1.2 mg/mL to 2 mg/mL.

The composition may optionally comprise an amino acid. As used herein, “amino acid” refers to a compound comprising a general structure:

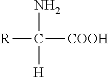

wherein R comprises a hydrogen, a substituted or unsubstituted alkyl group, a substituted or unsubstituted aryl group, and/or a heteroatom. Suitable amino acids comprise arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, and/or selenomethionine. The amino acid may comprise the L-enantiomer and/or the R-enantiomer.

The composition may comprise the amino acid in an amount of at least 20 mg/mL, such as at least 25 mg/mL, such as at least 30 mg/mL. The composition may comprise the amino acid in an amount of no more than 60 mg/mL, such as no more than 55 mg/mL, such as no more than 50 mg/mL. The composition may comprise the amino acid in an amount of 20 mg/mL to 60 mg/mL, such as 25 mg/mL to 55 mg/mL, such as 30 mg/mL to 50 mg/mL. The composition may comprise a first amino acid, a second amino acid, etc., in varying amounts within the ranges disclosed above, such that the total amount of amino acids is within the ranges disclosed herein.

The composition may optionally comprise a peptide. As used herein, “peptide” refers to a molecule comprising a short chain of amino acids that are linked by peptide bonds. The peptide may comprise an oligopeptide or a polypeptide. As used herein, “oligopeptide” refers to a peptide comprising 2 to 20 amino acid residues. As used herein, “polypeptide” refers to a peptide comprising greater than 20 to less than 50 amino acid residues. Examples of peptides that may be used in the compositions disclosed herein include but are not limited to collagen peptides, antimicrobial peptides, creatine peptides, copper peptides, ipamorelin, follistatin, BPC-157, and combinations thereof.

The composition optionally may comprise a vitamin or a combination of vitamins. As used herein, “vitamin” refers to a molecule essential to normal metabolic functioning that cannot be synthesized naturally by the subject. To the extent the vitamin may act as an antioxidant, for purposes of this disclosure, the vitamin is an additional component to the antioxidant. The vitamin or combination of vitamins may comprise vitamin A, vitamin B₁, vitamin B₂, vitamin B₃, vitamin B₄, vitamin B₅, vitamin B₆, vitamin B₇, vitamin B₈, vitamin B₉, vitamin B₁₀, vitamin B₁₁, vitamin B₁₂, vitamin C, vitamin D, vitamin E, and/or vitamin K. Vitamin C may comprise ascorbyl palmitate.

The composition may comprise the vitamin in an amount of at least 5 mg/mL, such as at least 10 mg/mL, such as at least 15 mg/mL. The composition may comprise the vitamin in an amount of no more than 35 mg/mL, such as no more than 30 mg/mL, such as no more than 25 mg/mL. The composition may comprise the vitamin in an amount of 5 mg/mL to 35 mg/mL, such as 10 mg/mL to 30 mg/mL, such as 15 mg/mL to 25 mg/mL.

The composition may optionally comprise a mineral. The mineral may comprise selenium, sulfur, iron, chlorine, cobalt, copper, manganese, molybdenum, iodine, and/or zinc.

The composition may comprise a first mineral, a second mineral, or more.

The composition may comprise the mineral in an amount of at least 3 mg/mL, such as at least 5 mg/mL, such as at least 6 mg/mL. The composition may comprise the mineral in an amount of no more than 20 mg/mL, such as no more than 15 mg/mL, such as no more than 12 mg/mL. The composition may comprise the mineral in an amount of 3 mg/mL to 20 mg/mL, such as 5 mg/mL to 15 mg/mL, such as 6 mg/mL to 12 mg/mL.

The mineral may comprise zinc. The composition may comprise zinc in an amount of at least 3 mg/mL, such as at least 4 mg/mL, such as at least 5 mg/mL. The composition may comprise zinc in an amount of no more than 15 mg/mL, such as no more than 12 mg/mL, such as no more than 10 mg/mL. The composition may comprise zinc in an amount of 3 mg/mL to 15 mg/mL, such as 4 mg/mL to 12 mg/mL, such as 5 mg/mL to 10 mg/mL.

The mineral may comprise a second mineral in addition to zinc. The second mineral may comprise selenium. The composition may comprise the second mineral in an amount of at least 0.8 mg/mL, such as at least 1.0 mg/mL, such as at least 1.2 mg/mL. The composition may comprise the second mineral in an amount of no more than 2.4 mg/mL, such as no more than 2.2 mg/mL, such as no more than 2 mg/mL. The composition may comprise the second mineral in an amount of 0.8 mg/mL to 2.4 mg/mL, such as 1.0 mg/mL to 2.2 mg/mL, such as 1.2 mg/mL to 2 mg/mL.

The composition may optionally further comprise an anti-inflammatory agent. The anti-inflammatory agent may comprise hyaluronic acid, curcumin, methotrexate, tofacitinib, 6-mercaptopurine, azathioprine, sulfasalazine, mesalazine, olsalazine, chlorophane/hydroxychloroquine, penicillamine, aurothiomalate, azathioprine, colchicine, corticosteroids, a beta-2 adrenoreceptor agonist (such as salbutamol, terbutaline, or salmeterol), a xanthine (such as theophylline or aminophylline), cromoglycate, nedocromil, ketotifen, ipratropium, oxitropium, cyclosporin, FK506, rapamycin, mycophenolate mofetil, leflunomide, ibuprofen, naproxen, a corticosteroid, such as prednisolone, a phosphodiesterase inhibitor, an adenosine agonist, an antithrombotic agent, a complement inhibitor, an adrenergic agent, an agent that interferes with signaling by proinflammatory cytokines such as TNF or IL-1 (e.g., an NIK, IKK, p38, or MAP kinase inhibitor), an IL-1 converting enzyme inhibitor, a T-cell signaling inhibitor (e.g., a kinase inhibitor), a metalloproteinase inhibitor, sulfasalazine, a 6-mercaptopurine, an angiotensin converting enzyme inhibitor, a soluble cytokine receptor (e.g., soluble p55 or p75 TNF receptors and the derivatives p75TNFRigG (etancercept) and p55TNFRigG (lenercept), siL-1RI, siL-1RII, siL-6R), an anti-inflammatory cytokine (e.g., IL-4, IL-10, IL-11, IL-13, or TGF), celecoxib, folic acid, hydroxychloroquine sulfate, rofecoxib, etanercept, infliximab, adalimumab, certolizumab, tocilizumab, abatacept, valdecoxib, sulfasalazine, methylprednisolone, meloxicam, methylprednisolone acetate, gold sodium thiomalate, aspirin, triamcinolone, acetonide, propoxyphene napsylate/apap, folate, nabumetone, diclofenac, piroxicam, etodolac, diclofenac sodium, oxaprozin, diclofenac sodium/misoprostrol, anakrina, salsalate, sulindac, and/or cyanocobalamin/fa/pyridoxine.

The composition optionally may further comprise an essential oil. As used herein, “essential oil” refers to a natural oil containing volatile chemical compounds from plants, which may be extracted from, for example, the flowers, seeds, fruits, roots, bark, and/or sap of a plant. Examples of essential oils that may be used in the present disclosure comprise sage oil, coriander oil, thyme oil, pimento berries oil, rose oil, anise oil, balsam oil, bergamot oil, rosewood oil, camphor oil, cardamom oil, cedar oil, cedar leaf oil, chamomile oil, cinnamon oil, sage oil, clary sage oil, clove oil, clove leaf oil, cypress oil, eucalyptus oil, fennel oil, fistree oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, menthol, neroli oil, orange oil, patchouli oil, pepper oil, black pepper oil, petitgrain oil, pine seed oil, pine needle oil, rosehip oil, rose otto oil, rosemary oil, sandalwood oil, spearmint oil, spikenard oil, vetiver oil, walnut oil, whitepine oil, wintergreen oil, and/or ylang.

The composition may optionally comprise additional components, such as ascorbic acid, ascorbic acid derivatives, glucosamine ascorbate, arginine ascorbate, lysine ascorbate, nictinamide ascorbate, niacin ascorbate, allantoin ascorbate, creatine ascorbate, creatinine ascorbate, chondroitin ascorbate, chitosan ascorbate, DNA ascorbate, carnosine ascorbate, tocotrienol, rutin, hesperidin, diosmin, mangiferin, mangostin, cyanidin, astaxanthin, lutein, lycopene, resveratrol, tetrahydrocurcumin, rasmarinic acid, hypericin, allegic acid, chlorogenic acid, oleuropein, α-lipoic acid, niacinamide, lipoate, andrographolide, carnosine, niacinamide, potentilla erecta extract, polyphenols, grape seed extract, pyncnogenol, pyridoxine, magnolol, honokiol, paeconol, resacetophenone, quinacetophenone, arbutin, and/or kojic acid.

The composition may be formulated as a nanoemulsion. As used herein, “nanoemulsion” refers to a dispersion of droplets of one fluid within another fluid. The droplets comprise a particle size of 30 nm to 100 nm. Particle size may be measured as described above.

The composition may be formulated as a topical composition. As used herein, “topical composition” refers to a composition that is formulated to be applied to the surface of the skin. The topical composition may optionally comprise exfoliating agents. The exfoliating agents may comprise a chemical exfoliant or exfoliant particles. Examples of suitable exfoliating agents include minerals, organic particles, water-swellable pulverulent polymers (powders or beads), polyethylene particles (powders or beads), jojoba spheres, ground shells of fruit stones, pumice stone, glass beads, and/or aluminum oxide. The topical composition may be in the form of a cream, a liniment, a balm, a lotion, a gel, or an ointment.

The topical composition may comprise additional skin care additives. Suitable skin care additives include but are not limited to cleaning agents, skin conditioning agents, perfumes, sunscreens and/or sunblocking compounds, pigments, moisturizers, detergents, thickening agents, emulsifiers, humectants, emollients, deodorant actives, dermatologically acceptable carriers, and/or surfactants. The topical composition may further comprise additional components, such as skin soothing agents, skin smoothing agents, healing agents, anti-aging agents, skin moisturizing agents, anti-wrinkle agents, anti-atrophy agents, antimicrobial agents, anti-inflammatory agents, anti-pruriginous agents, external anesthetic agents, antiviral agents, keratolytic agents, free radical scavengers, antiseborrheic agents, depigmentating or propigmenting agents, antiglycation agents, tightening agents stimulating the synthesis of dermal or epidermal macromolecules and/or preventing their degradation, anticellulite agents, slimming agents, agents that may act on microcirculation, agents that may act on the metabolism, cosmetic astringents, anti-acne agents, anti-caking agents, anti-foaming agents, buffering agents, bulking agents, chelating agents, chemical additives, pH adjusters, pH regulators, sequestrants, skin bleaching and lightening agents, skin tanning agents, preservatives, colorants, and/or dyes.

The compositions disclosed herein may be formulated as a tincture. As used herein, “tincture” refers to a composition formed by soaking plant parts in a liquid to form a liquid infused with plant extracts. Tinctures provide a method for oral administration of the plant extract. Tinctures are prepared by mixing the plant parts with a suitable solvent wherein a component or components of the plant part are extracted into the solvent. Suitable tincture solvents used in the present disclosure include the carriers disclosed herein.

The composition disclosed herein may comprise an Echinacea extract; a policosanol; glutathione; quercetin; zinc; selenium; ascorbyl palmitate; lysine; olive oil; avocado oil; and water. The composition may comprise the Echinacea extract in an amount of 5 mg/mL to 35 mg/mL; the policosanol in an amount of 5 mg/mL to 35 mg/mL; the glutathione in an amount of 20 mg/mL to 60 mg/mL; the quercetin in an amount of 0.8 mg/mL to 2.4 mg/mL; the zinc in an amount of 3 mg/mL to 15 mg/mL; the selenium in an amount of 0.8 mg/mL to 2.4 mg/mL; the ascorbyl palmitate in an amount of 5 mg/mL to 35 mg/mL; the lysine in an amount of 20 mg/mL to 60 mg/mL; the olive oil in an amount of 400 mg/mL to 850 mg/mL; the avocado oil in an amount of 3 mg/mL to 15 mg/mL; and/or the water in an amount of 200 mg/mL to 600 mg/mL.

### Methods

The present disclosure is further directed to a method of treating a disorder comprising administering a therapeutically effective amount of one of the compositions disclosed herein. Those in need of treatment may include those already with a disease, disorder or illness and those in which a disease, disorder, or illness is to be prevented.

A person of ordinary skill understands that various factors influence the amount required to treat a patient effectively, and that accordingly the dosage and administration may be chosen in view of the patient to be treated and may be adjusted for sufficient levels of the active agent(s) or to maintain the desired effect. Factors that may be taken into account include but are not limited to the severity of the disease state, e.g., intermediate or advanced stage of disease; age, weight, gender, and overall health of the patient; diet, time, and frequency of administration; form of iron deficiency; route of administration; drug combinations; reaction sensitivities; prior treatments; and tolerance/response to therapy.

The compositions disclosed herein may be administered to treat various disorders, including but not limited to diabetes; osteoporosis or low bone density; inflammatory disorders such as arthritis; kidney disease; herpes simplex virus 1 and 2; shingles; hypertension; viral infections such as viral pneumonia; bacterial infections such as bacterial pneumonia and S. pyrogenes infections; high cholesterol; high triglyceride levels; and metabolic disease. Administration of the compositions disclosed herein may also result in improved immune functioning, enhanced metabolism, and accelerated healing time of, for example, cuts, bruises, broken bones, tendon and ligament damage, sprains and strains, and the like. Administration of the compositions disclosed herein may also result in anti-aging effects, including but not limited to the reduction in the appearance of fine lines and wrinkles, improved liver function, and improved blood vessel flexibility.

It has been surprisingly discovered that the compositions disclosed herein regulate blood glucose levels in patients diagnosed with diabetes.

The composition may be administered by any route in order to perform the intended function, including but not limited to orally, topically, and/or subcutaneously. Oral administration may comprise sublingual administration.

At least 1 drop of a tincture comprising one of the compositions disclosed herein may be administered, such as at least 3 drops, such as at least 5 drops. No more than 20 drops of a tincture comprising one of the compositions disclosed herein may be administered, such as no more than 15 drops, such as no more than 10 drops. 1 to 20 drops of a tincture comprising one of the compositions disclosed herein may be administered, such as 3 to 15 drops, such as 5 to 10 drops.

When administered orally, such as sublingually, the composition may be administered in a dose of at least 50 μL, such as 50 μL to 1.0 mL, such as 50 μL to 0.75 mL, such as 50 μL to 0.5 mL, such as 50 μL to 0.25 mL, such as 50 μL to 200 μL, such as 50 μL to 150 μL. The composition may be administered at a dose of 2 mg of the composition per 1 kg of the subject's weight to 500 mg of the composition per 1 kg of the subject's weight, such as 2 mg/kg to 350 mg/kg, such as 2 mg/kg to 200 mg/kg, such as 2 mg/kg to 100 mg/kg, such as 2 mg/kg to 50 mg/kg.

The composition may alternatively be orally administered by a tablet or capsule comprising the composition. The tablet or capsule may comprise a dosage of the composition of at least 20 mg, such as at least 30 mg, such as at least 40 mg. The tablet or capsule may comprise a dosage of the composition of no more than 200 mg, such as no more than 150 mg, such as no more than 100 mg. The tablet or capsule may comprise a dosage of the composition of 20 mg to 200 mg, such as 30 mg to 150 mg, such as 40 mg to 100 mg.

The methods of treating described herein may be repeated 1, 2, 3, 4, or more times per day, resulting in a daily administration of a total daily dose of 50 μL to 4.0 mL per day. Single or multiple administrations per day can be carried out each day or 2, 3, 4, or 5 times per week for 1 month or more, 2 months or more, 3 months or more, 1 year or more, or throughout the lifetime of the patient.

### Kits

The compositions described herein may be included in a kit, pack, or dispenser for treating a disease or disorder, referred to collectively herein as a “kit,” optionally with instructions for administration of such compositions. A kit may include one of the compositions disclosed herein. Optionally, additional compositions also may be included. The kit also may include a pharmaceutically acceptable carrier suitable for each composition included therein. The compositions and carrier(s) may be housed in vials or other suitable containers. The compositions may be lyophilized, resuspended, liquid, powder, or in any other suitable form. The kit also may include an instrument for administration, such as a syringe, applicator, and the like.

The kit may include instructions recorded on any recording medium known to those skilled in the art, and may set forth instructions for reconstituting the compositions contained in the kit and/or for practicing the methods disclosed herein. Optionally, the instructions may include instructions to download or otherwise access instructions that are remotely stored. In examples, the recording medium may include, but is not limited to, a kit insert, a label on one or more of the containers housing the compositions or carriers, or may be stored on any computer readable storage medium. The instructions may be stored remotely in downloadable or non-downloadable form, accessible, for example, via the internet.

Illustrating the disclosed subject matter are the following examples that are not to be considered as limiting the disclosure to their details. All parts and percentages in the examples, as well as throughout the specification, are by weight unless otherwise indicated.

### EXAMPLES

For all examples, a tincture was administered comprising the composition set forth in Table 1.

TABLE 1

| | Dosage¹ |
|---|---|
| Component | (mg) |
| Olive Oil | 162 |
| Water | 108 |
| Policosanol | 4.77 |
| Glutathione | 9.91 |
| L-Lysine | 9.91 |
| Echinacea | 5.04 |
| Ascorbyl Palmitate | 5.04 |
| Selenium | 0.40 |
| Quercetin | 0.40 |
| Zinc | 1.80 |
| Avocado Oil | 1.67 |

¹Dosages are per 0.25 mL of composition

### Example 1

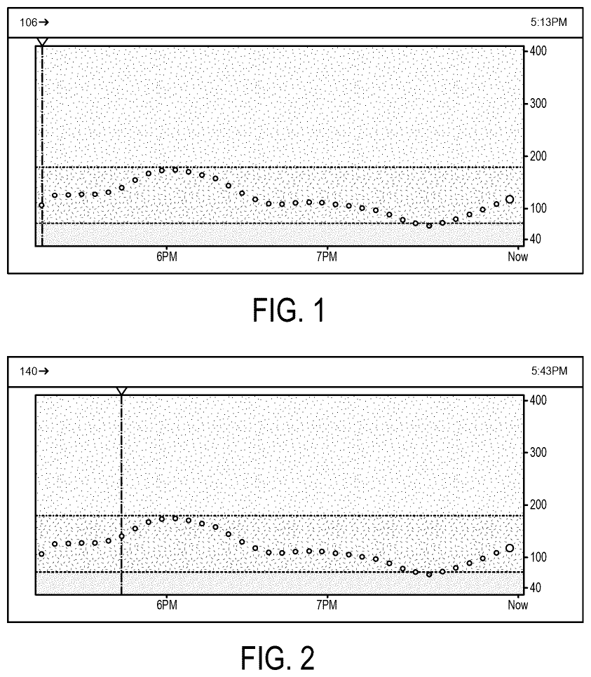

Patient 1 sublingually self-administered 5 drops of the tincture and half a normal insulin dose at 5:13 PM. FIG. 1 is a graph of Patient 1's blood glucose as a function of time, with the vertical line indicating the time at which the tincture was self-administered. As shown in FIG. 1, blood glucose was already slightly rising prior to administration of the tincture.

At 5:43, Patient 1 began to eat dinner. FIG. 2 shows a graph of Patient 1's blood glucose as a function of time, with the time at which Patient 1 began eating dinner demarked by a vertical line. As shown in FIG. 2, Patient 1's blood glucose never went over 180 mg/dL, which is considered high blood glucose.

### Example 2

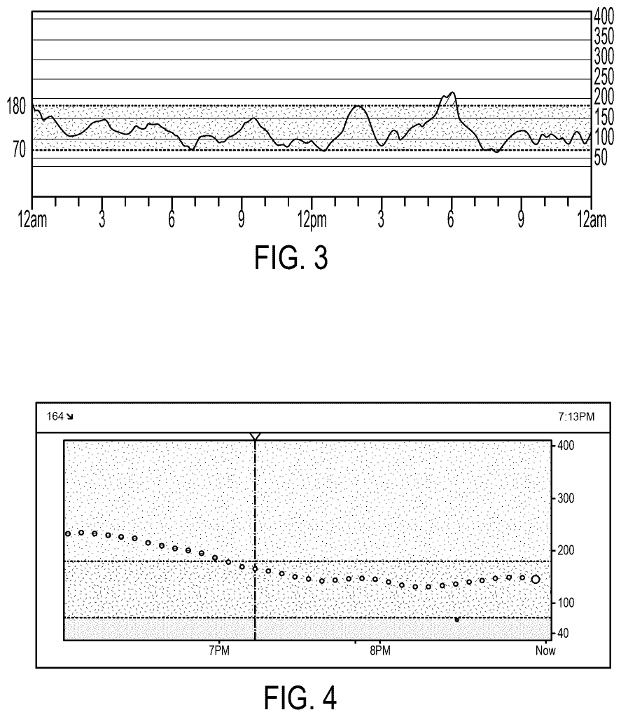

Patient 1 sublingually self-administered 5 drops of the tincture and a half dose of insulin at 7:30 AM on a different day. Patient 1 ate breakfast at approximately 8:00 AM. Patient 1's blood glucose levels never rose above 180 mg/dL following breakfast. A blood glucose level above 180 mg/mL was not measured until approximately 2:00 PM-6 hours following tincture administration. A graph of Patient 1's blood glucose levels as a function of time over a 24-hour period is provided in FIG. 3.

### Example 3

Patient 2 typically required pre-dosing of insulin at least 20 minutes prior to meals to avoid blood sugar spikes. Patient 2 self-administered half of the typical dose of insulin and sublingually self-administered 5 drops of the tincture. Three hours after eating, the patient's blood glucose level was 89 mg/dL. FIG. 4 provides a graph of the patient's blood glucose as a function of time. The vertical line demarks when Patient 2 self-administered the insulin and tincture.

### Example 4

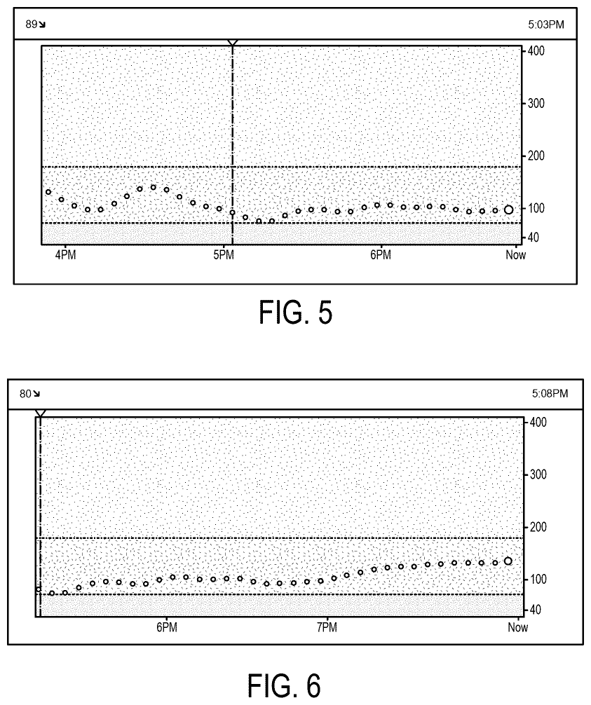

Patient 2 sublingually self-administered 5 drops of the tincture at approximately 5:03 PM. Patient 2 took half the typical dose of insulin 35 minutes after dinner. Three hours after eating, the patient's blood glucose level was 95 mg/dL. Results are provided in FIG. 5, in which the vertical line demarks when tincture was self-administered.

### Example 5

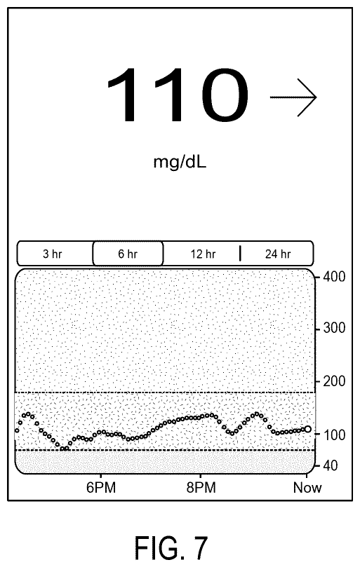

Patient 2 self-administered half a dose of insulin and sublingually self-administered 5 drops of the tincture after eating dinner. Patient 2's blood glucose peaked at 160 mg/dL, then dropped to 110 mg/dL. Graphs showing Patient 2's blood sugar levels as a function of time are provided in FIGS. 6 and 7. The vertical line in FIG. 6 demarks when the tincture was self-administered.

### Example 6

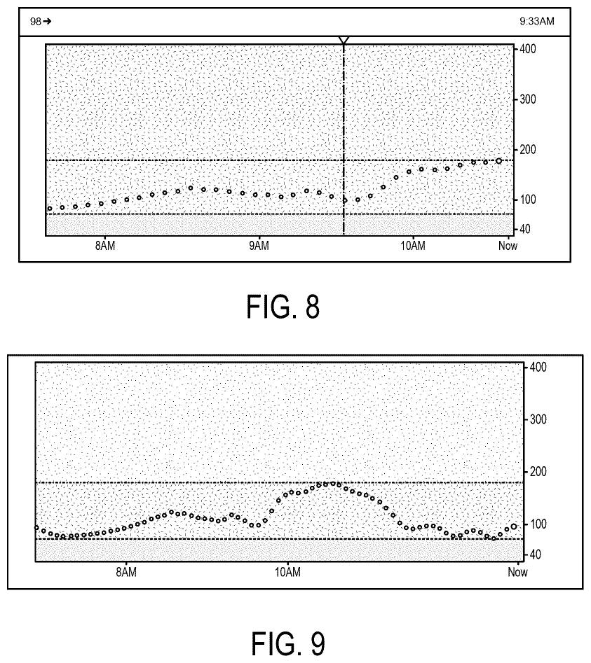

Patient 2 sublingually self-administered 5 drops of the tincture at 9:33 AM. After eating breakfast, the patient self-administered a dose of insulin that was 25% less than the typical dose. Patient 2's blood glucose peaked to approximately 178 mg/dL, but immediately dropped back down. Patient 2 had to ingest juice three times in order to stay above 80 mg/dL. FIGS. 8 and 9 provide graphs of Patient 2's blood glucose levels as a function of time. The vertical line in FIG. 8 demarks when the tincture was self-administered.

### Example 7

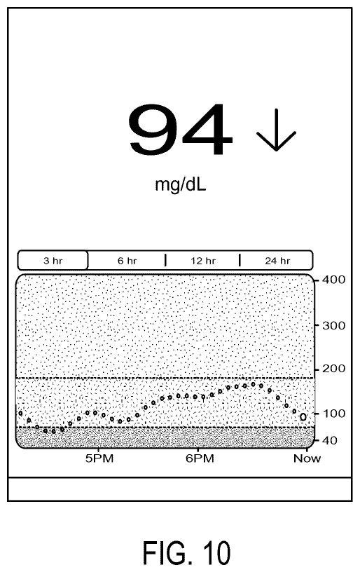

Patient 3 sublingually self-administered 5 drops of the tincture at 5:00 PM, contemporaneous with eating dinner. Patient 3's blood glucose level was 64 mg/dL at 7:03 PM. A graph showing Patient 3's blood glucose levels as a function of time is provided in FIG. 10. Table 2 provides measurements of Patient 3's blood glucose levels at various times.

TABLE 2

| Time | Blood Glucose (mg/dL) |
|---|---|
| 7:03 PM | 94 |
| 7:18 PM | 81 |
| 7:28 PM | 87 |
| 7:33 PM | 92 |
| 8:03 PM | 113 |

We claim:

1. A composition comprising:
an Echinacea extract;
a long chain alcohol; and
a carrier.

2. The composition of claim 1, wherein the Echinacea extract is derived from Echinacea purpurea, Echinacea angustifolia, and/or Echinacea pallida.

3. The composition of claim 1, wherein the Echinacea extract is derived from roots, leaves, and/or flowers of an Echinacea plant.

4. The composition of claim 1, comprising:
the Echinacea extract in an amount of 5 mg/mL to 35 mg/mL;
the long chain alcohol in an amount of 5 mg/mL to 35 mg/mL; and/or
the carrier in an amount of 600 mg/mL to 1,500 mg/mL.

5. The composition of claim 1, wherein the long chain alcohol comprises octacosanol, hexacosanol, and/or triacontanol.

6. The composition of claim 1, wherein the carrier comprises a phospholipid and/or water.

7. The composition of claim 6, wherein the composition comprises:
the phospholipid in an amount of 400 mg/mL to 850 mg/mL; and/or
the water in an amount of 200 mg/mL to 650 mg/mL.

8. The composition of claim 1, wherein the composition further comprises an antioxidant source in addition to the Echinacea extract, an ionophore, an amino acid, a vitamin, and/or a mineral.

9. The composition of claim 8, wherein the composition comprises:
the antioxidant source in an amount of 20 mg/mL to 60 mg/ml;
the ionophore in an amount of 0.8 mg/mL to 2.4 mg/mL;
the amino acid in an amount 20 mg/mL to 60 mg/ml;
the vitamin in an amount of 5 mg/mL to 35 mg/mL; and/or
the mineral in an amount of 3 mg/mL to 20 mg/mL.

10. The composition of claim 9, wherein:
the antioxidant source comprises glutathione;
the ionophore comprises quercetin;
the amino acid comprises lysine and/or selenomethionine;
the vitamin comprises a C vitamin; and/or
the mineral comprises selenium and/or zinc.

11. A method of treating a disorder comprising:
administering to a subject in need thereof a therapeutically effective amount of the composition of claim 1.

12. The method of claim 11, wherein the composition is administered sublingually, orally, or topically.

13. The method of claim 11, wherein the composition is administered at a total daily dose of 50 μL to 4.0 mL.

14. The method of claim 11, wherein the composition is administered at a dose of 2 mg of the composition per 1 kg of the subject's weight to 500 mg of the composition per 1 kg of the subject's weight.

15. The method of claim 11, wherein the disorder comprises diabetes.

16. A kit for the treatment of a disease, comprising the composition of claim 1 and instructions for administering the composition of claim 1 to a subject.

17. The kit of claim 16, wherein the instructions include instructions for administering a second composition to the subject prior to the composition of claim 1, after the composition of claim 1, or contemporaneously with the composition of claim 1.

18. The kit of claim 17, wherein the second composition comprises insulin.

19. The kit of claim 17, further comprising the second composition.

20. The kit of claim 16, wherein the instructions instruct administering the composition of claim 1 to the subject at a dose of 2 mg of the composition per 1 kg of the subject's weight to 500 mg of the composition per 1 kg of the subject's weight.
